(19) United States
(12) Patent Application Publication
(10) Pub. No.: US 2013/0109980 A1
(43) Pub. Date: May 2, 2013

(54) SYSTEMS AND METHODS FOR A WIRELESS VASCULAR PRESSURE MEASUREMENT DEVICE

(71) Applicant: Tat-Jin Teo, Santa Clara, CA (US)

(72) Inventor: Tat-Jin Teo, Santa Clara, CA (US)

(21) Appl. No.: 13/664,357

(22) Filed: Oct. 30, 2012

Related U.S. Application Data
(60) Provisional application No. 61/554,227, filed on Nov. 1, 2011.

(51) Int. Cl. A61B 5/0215

(52) U.S. Cl. 600/488; 600/486

(57) ABSTRACT

A vascular measuring system includes an elongated sleeve configured to be delivered over a standard guide wire configured to be threaded into a vascular pathway of the human, and includes sensor(s) coupled to the sleeve. The sensor(s) measure physiological parameter(s) of the human. Alternatively, the sensor(s) may be located at the end a guide wire without a sleeve. The system may include a connector coupled to the sleeve or guide wire, and receives the measured parameter(s) from the sensor(s), and display the result of processed parameter(s).

### CROSS REFERENCE TO RELATED APPLICATIONS

This application claims priority to U.S. Provisional Application Ser. No. 61/554,227 filed on Nov. 1, 2011, entitled “Systems and Methods for a Wireless Vascular Pressure Measurement Device”, which is hereby fully incorporated by reference.

### BACKGROUND

The present invention relates to a system and methods for a vascular pressure measurement device.

Pressure wire has been used in the catheterization laboratory as part of the Percutaneous Coronary Intervention (PCI) procedure since the late 1980's. The form factor most commonly used is that of the 0.014″ diameter guide wire.

A typical construction of a pressure wire involves a radio opaque spring tip in the distal end, a housing or holder for the pressure sensor itself a few centimeters proximal to the distal end and a lumen, which is a hollow channel, to accommodate the electrical conductors or optical fibers depending on whether the pressure sensor is electrical or optical in its theory of operation.

At the proximal end where the pressure wire exits the human body, an electrical interface is typically provided for signal acquisition, processing and display. Some user input interface can also be provided.

There are times the pressure wires are used like a guide wire on which other interventional device like balloon or stent deployment system can be delivered. Consequently, the profile of a pressure wire needs to be maintained throughout the length of the body of the pressure wire. This requirement also applies to the electrical contacts where the above electrical interface for acquisition is located. Having electrical contacts that remain flushed with the pressure wire body profile is therefore important.

The electrical interface where the pressure signal is acquired and/or processed also needs to be removable when the pressure wire is to be used as a guide wire for delivery of other interventional devices.

Some clinicians, for tactile familiarity, have a preference to use a particular guide wire to begin the interventional procedure. These guide wires are also referred to as the primary guide wires. If a separate pressure wire is used for subsequent pressure measurement, it would then involve a wire exchange step which is sometimes undesirable especially if it is a very difficult lesion, a narrowing or obstruction, in the vessel, to cross the first time.

It would then be preferable to measure the pressure with a catheter over the guide wire that is already in place. The disadvantage is that the accuracy of the pressure measurement relative to that from a pressure wire might be reduced due to the presence of the catheter. It is therefore important to have a micro-catheter as small as possible.

The trade off between measuring the pressure in the form of a guide wire or a stand-alone micro-catheter will be discussed when the present invention is further described below.

While the sensing technology continues to make progress in terms of sensor miniaturization and improved processing and manufacturing method can achieve better performance and cost, many limitations remain.

Some of the limitations of prior art pressure wire are described here.

It is common to have a lumen in the region proximal to the sensor to accommodate the electrical or optical transmission lines. Unfortunately, this reduces somewhat the ability to provide a 1:1 torque transmission from the proximal end to the distal end of the pressure wire. Consequently, many physicians tend to use their preferred guide wire to cross the lesion in the vessel and only when they want to perform pressure measurement, they would do a wire exchange to deploy a pressure wire.

A culprit lesion that is responsible for the symptoms that bring the patient into the catheterization laboratory in the first place is often times one that has a severe narrowing of the vessel lumen. Many physicians may see no need to further measure the pressure gradient caused by that culprit lesion to assess its hemo-dynamic significance. In addition, it would be challenging to deploy a pressure wire there since it usually will not perform as well as one designed to be a primary guide wire.

On the other hand, if there are multiple lesions, one may appear to be only marginally constrictive from the appearance of the angiogram. The decision to intervene will then be based upon the hemo-dynamic of the lesion and pressure gradient measurement will be very helpful.

The pressure wire is also tethered to a non-sterile electronic equipment which as described above will acquire and process the signal from the sensor. The electronic equipment typically will also have a user input device to facilitate the procedure and provide a display for the signal as well as any processed results.

This need for electronic equipment near the sterile field in the catheterization laboratory can impede a smooth work flow in the catheterization laboratory. One solution is to have the electrical interface located far enough from the sterile field to avoid accidental contamination. However, this arrangement comes at the expense of degraded signal quality due to the parasitic noise induced by the extended connection length.

U.S. Pat. No. 7,724,148 provides a wireless interface which is attached at the proximal end of the pressure wire. Pressure signals are processed and transmitted from the proximal end of the pressure wire wirelessly to a wireless receiver in the non-sterile area. The size is such that while it can function as a handle for the pressure wire, it is too large to function adequately like a torque device, known sometimes as a torquer, commonly used to manipulate a 0.014″ guide wire.

The position of the wireless transceiver is also fixed by the location of the electrical contacts on the pressure wire and would not allow the operator to manipulate the guide wire in a way that is similar to a torque device. A regular torque device can be used at an arbitrary position along the proximal region of the guide wire according to personal preference and the requirement of the anatomy involved at the procedure.

Implementing the wireless transceiver in the form factor of a torque device allows it to move to a location along the guide wire closer to where it enters the touhy borst. This will allow better control of the wire movement.

With prior art pressure wire, it is common to have only a single sensor at the distal tip as described above. In some procedure, it is desired to measure both the pressure distal to the lesion in a coronary vessel as well as the pressure in the aorta, the ratio of which is a useful ratio to estimate a parameter known as Fractional Flow Reserve

This desire to measure pressures at two locations requires a pullback operation to move the sensor from a location distal to a lesion in a coronary vessel to a location proximal to the lesion. Having multiple sensors would typically increase the number of transmission lines and can be a difficult task given the small space of a guide wire form factor.

It is therefore apparent that an urgent need exists for an improved pressure measuring device that includes one or more of the following improvements: (i) elimination of the hollow lumen in the body of the guide wire, (ii) wireless transmission, (iii) multiple sensors and (iv) stand-alone micro-catheter compatible with primary guide wires, resulting in better handling characteristics, better measurements, and shortened invasive procedures.

### SUMMARY

To achieve the foregoing and in accordance with the present invention, a system and method for measuring at least one physiological parameter of at least one location inside a human is provided. In particular, a wireless vascular pressure measurement device for measuring parameter(s) at one or more vascular locations inside a human is provided.

In one embodiment, the vascular measuring system includes an elongated sleeve configured to be delivered over a standard guide wire configured to be threaded into a vascular pathway of the human, and may include at least one sensor operatively coupled to a distal end of the sleeve, wherein the at least one sensor is configured to measure the at least one physiological parameter of the human.

In some embodiments, the at least one sensor may be located at the distal end of a guide wire without a sleeve. The system may include a connector operatively coupled to the proximal end of the sleeve or guide wire. The connector is configured to receive the at least one measured parameter from the at least one sensor, and display the result of processing from the at least one parameter from the at least one location. The connector may also include a wireless transmitter and is adequately light weight such that it can be attached onto the guide wire in substantially arbitrary location along a proximal portion of the guide wire thereby enabling the connector to function like a torque device for manipulating the guide wire inside the vascular pathway of the human.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1 is a schematic showing the key components making up a pressure wire measurement system;

FIG. 2 is a schematic showing the conductors between the sensor and proximal electrical contacts in a prior art embodiment;

FIG. 3 illustrates one preferred embodiment of the electrically conductive structures of the present invention;

FIG. 4 illustrates the cross-sectional view of FIG. 3;

FIGS. 5a, b, c and d illustrate a torque device in accordance to one embodiment of the invention;

FIG. 6 illustrates one preferred embodiment of the pressure wire to provide a guiding mechanism so that the torque device will engage the conductive traces at the appropriate orientation;

FIG. 7 illustrates another preferred embodiment of the pressure wire measurement system where there are two sensors deployed on a sleeve that can be delivered over a traditional guide wire, 110, not shown, and a torque device can wirelessly activate the sensors and shows the results from the signals return by these two sensors; and

FIG. 8 illustrates another embodiment where the stand alone sleeve catheter with two sensors is in a rapid exchange catheter configuration with guide wire, 110, and a catheter handle, 810, now serving as the display for either the waveforms from the two sensors or the results after processing of the two waveforms or both, depending on the display size available. Two switches to control the electronics in the handle are also shown in this illustration.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

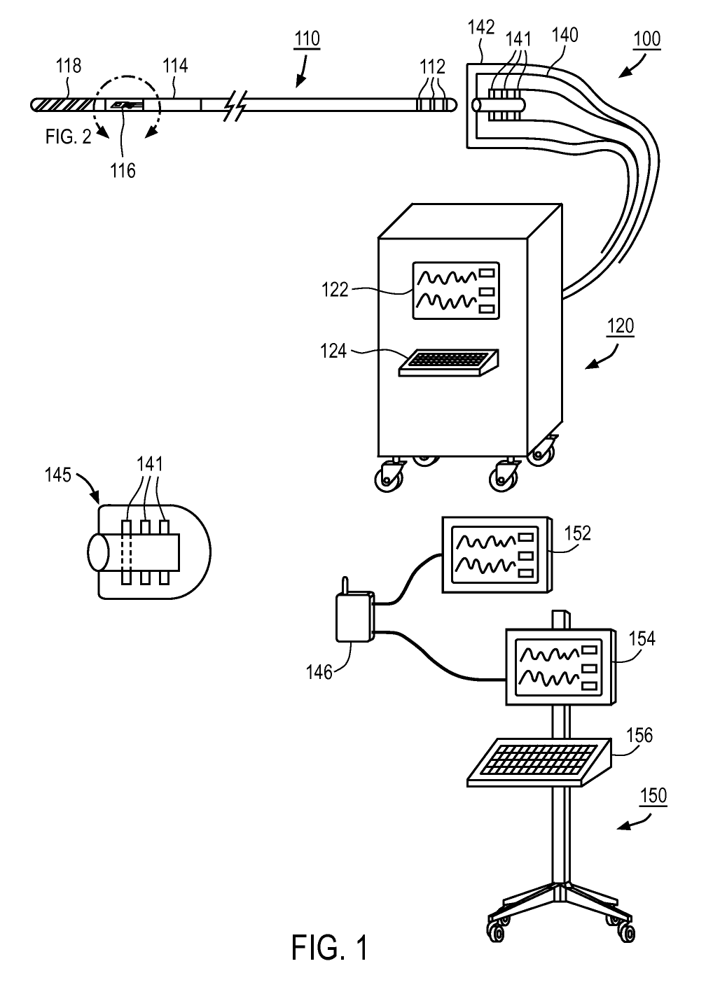

FIG. 1 shows one embodiment of a pressure wire measurement system, 100, not to scale. It includes a pressure wire, 110. The distal end, designated 118, is usually radio-opaque to allow for visualization under X-ray and is usually implemented as a coil to make it floppy and atraumatic. The pressure sensor is designated 116 and is often followed by another coil section 114 for desired stiffness. The remaining body of the pressure wire often has a hollow lumen to accommodate the electrical transmission lines (not shown) connecting the sensor 116 with the electrical contacts 112 at the proximal end.

The hollow lumen in the proximal portion of the pressure wire designed to accommodate the electrical or optical transmission conductors reduces the fidelity of the torque transmission due to the reduced rigidity of the body of the pressure wire. System 100 addresses this issue by having thin conductive traces on the central core wire.

FIG. 1 also shows a connector 140 that couples to the proximal end of the pressure wire 110. Internal to connector 140, there are electrical contacts 141 that mate with the counterpart 112 on the pressure wire. The connector 140 being non-sterile needs to be enclosed with a sterile barrier, 142, typically a sterile bag, to prevent contamination of the sterile field during the PCI procedure.

It is also possible to have a long pressure wire such that the connect 140 is far remove from the sterile field where the risk of contamination is low and a sterile barrier 142 may not be needed. However, if long transmission lines are used as a consequence of having a long pressure wire, signal quality may be degraded.

The connector 140 is coupled to an electronic equipment, 120, where the signals from the sensor can be acquired, processed and display with the display 122. If user input is needed, an input device 124 can also be located on the electronic equipment 120.

In another embodiment, a wireless implementation is described. In this embodiment, a wireless transceiver 145 is coupled to the pressure wire such that the electrical contacts, 141, in the transceiver 145, mates with the electrical contact 112 on the pressure wire 110. The signals are then wirelessly received by a wireless transceiver 146 which can then display the information on a display 152 or couple to the electronic equipment 150 which may take the form of an Intravenous pole with a display 154 and an input device 156.

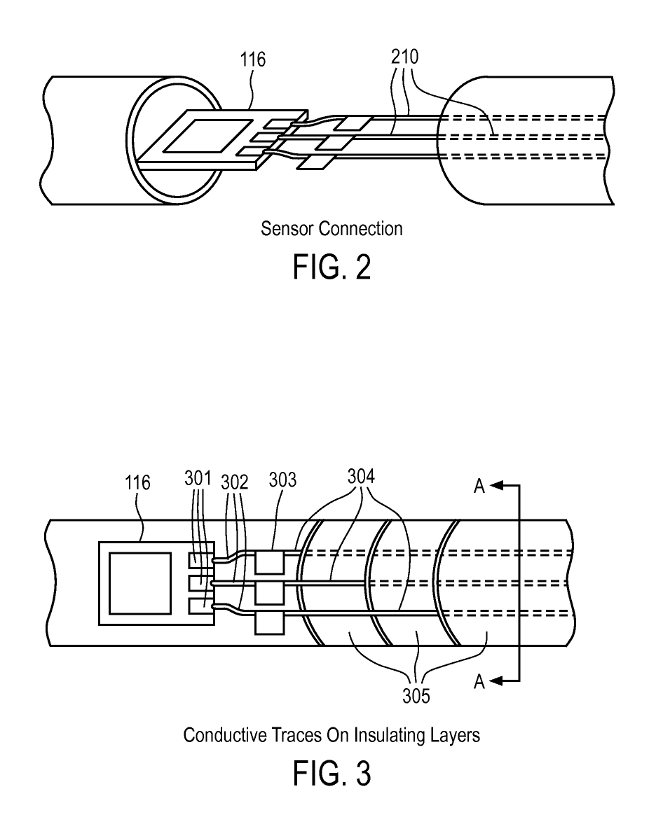

FIG. 2 shows a close up view of the sensor 116 with the electrical transmission conductors 210. These conductors terminate at the electrical contacts 112 at the proximal end of the pressure wire 110. With this construction, the mating connector, whether in the form of a connector 140, or in the form of a wireless transceiver 145 is located at the proximal end of the pressure wire 110 where the electrical contacts 112 are located on the pressure wire 110.

This arrangement for the wireless transceiver 145 can be an impediment to the work flow as transceiver should be smaller and light weight and ideally should perform like a torque device. A torque device, not shown, also needs to be able to be positioned anywhere proximal to where the pressure wire exits the human body and not be constrained to the proximal end or a particular fixed location.

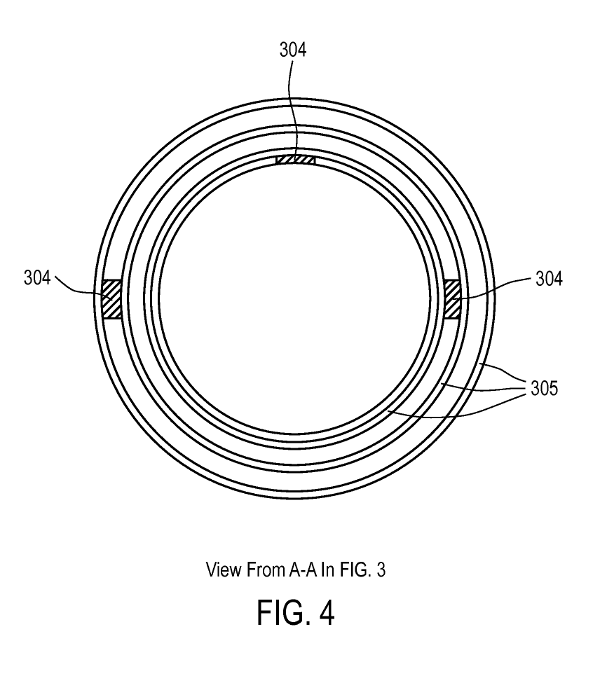

Referring to FIG. 3, the conductors that electrically connect the sensor to the equipment for acquisition, processing and display have been replaced with electrically conductive traces, 304, embedded in insulating layers, 305. Three such insulating layers are illustrated in FIG. 3.

In some embodiments, the traces are terminated in pads, 303, which are connected to pads, 301, on the sensor chip via wire bonding with gold wires, 302. Other connection schemes known to persons skilled in the art are also possible.

The traces 304 are distinguished from one another by the number of insulating layers 305 as well as the circumferential locations as indicated in the cross-sectional representation in FIG. 4.

Shielding layers, not shown, can also be implemented to improve the electrical performance of these conductive traces if needed.

These traces 304 can be metallization via various depositing process or conductive polymer and the insulating layers 305 can be various insulating polymers, like polyimide film.

It is also possible to print conductive polymer onto an insulating substrate and achieve similar results. Beside these additive processes, it is also possible to start with a conductive layer on top of an insulating layer, subtractive processes can then be used where the conductive material is removed to result in conductive traces remaining on the insulating layer to serve as conductors.

It is possible to have variations along this theme. For example, multiple conductive traces can reside in the same layer underneath one insulating layer if they can be separated adequately apart. This may be an advantage in the case of multiple sensor chips. One sensor chip can have its conductive traces residing in one layer, while the other can have its conductive traces in another layer.

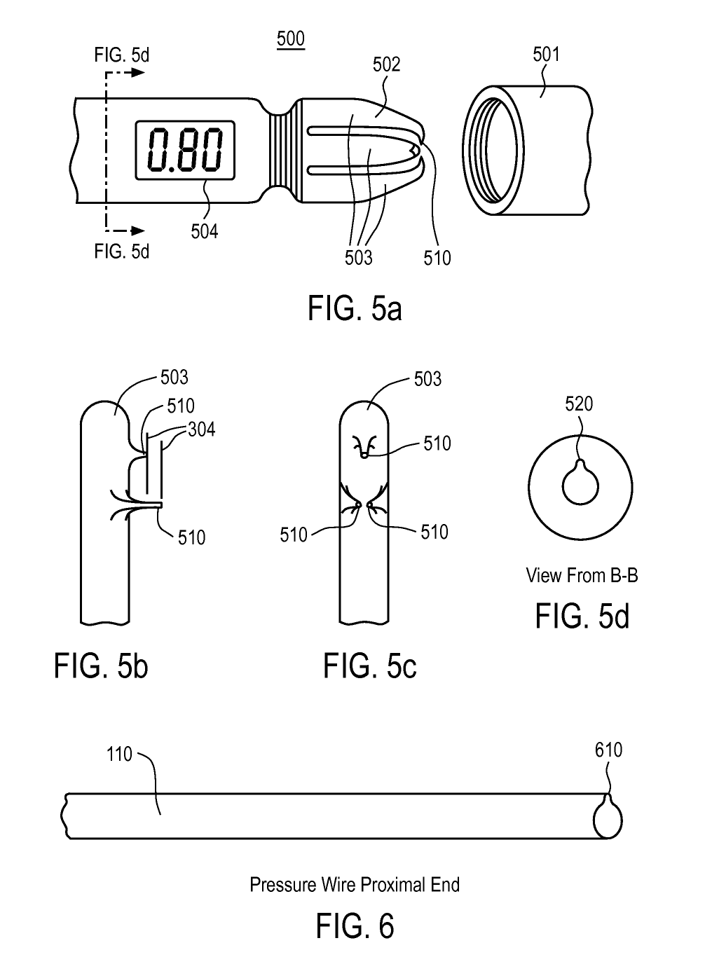

In FIG. 5a, an exemplary torque device 500, is shown with a cap 501 and collet 502, an arrangement where as the cap is advanced, the fingers 503 of the collet 502 will close on and grip on the pressure wire 110. Pressure wire 110 is not shown.

Different ways to implement a torque device are possible.

In FIG. 5a, some of the fingers have a tapered tip 510, capable of penetrating the insulation layers 305, and making contact with the appropriate traces 304, thereby forming electrical connection(s). Different shape and arrangement for the finger 503 to make electrical contacts with the conductive traces 304 are also possible.

Different fingers 503 can have different length tapered tip 510 capable of penetrating to the correct depth to make contact with the conductive trace 304 through the various insulating layers 305.

FIGS. 5b and 5c show two close up views of one embodiment of a finger with a tapered tip configuration designed to simultaneously penetrate two insulating layers 305 to make contact with conductive traces 304 lying at two different depths.

The configuration is such that while making contact with the deeper layer, it avoids shorting with the shallower layer.

This implementation of the tapered tips is useful where multiple sensor chips 116 are present at the distal end of the pressure wires and the conductive traces are embedded in separate layers at different depths. Different length tapered tip 510 can engage different sensor chip signals at different depth levels with no ambiguity. Even if the number of conductive traces is small enough to fit with in the circumference of a single layer of insulating layer, it may still be advantageous to keep the number of fingers 503 small but utilize multiple tapered tips 510 to engage the conductive traces at different depths. Such flexibility is provided for in these embodiments.

Other configurations and methods for the tapered tips to engage the conductive traces are also possible.

In FIG. 5d, a view from B-B of FIG. 5a, the body of the collet 502 has a guiding track 520 to guide the insertion of the torque device such that the orientation of the fingers 503 remain aligned with the conductive traces 305 correctly. In FIG. 6, the portion of the pressure wire 110 that accepts the torque device has a corresponding guiding ridge 610 that allows the torque device to slide along it once the guiding track 520 is aligned with the guiding ridge 610.

This is one example of a mechanical means to ensure a proper orientation of the torque device. Using a visible strip marking on the guide wire for aligning with a counterpart marking on the torque device is an example of a visual means for achieving correct alignment.

Other ways to provide orientation guidance are known for those skilled in the art.

In FIG. 5a, a display 504 is also shown, where result derived from the sensor can be made available to the user of the torque device.

This torque device being able to make electrical connection with the sensor 116 can now provide the needed signal acquisition, processing and wireless transmission to a receiver outside the sterile area of the catheterization laboratory.

In this embodiment, it is important to make the transceiver unit small and light weight as well as being able to position freely along a much larger range in the proximal portion of the pressure wire and behave like a torque device.

To achieve this behavior, some parts of the acquisition and processing are partitioned off the transceiver 145 and locate on the pressure wire body proper. The constraint is to maintain the profile such that the diameter of the entire pressure wire can still accept delivery of other device designed to be delivered over a guide wire, e.g. balloon and stent, usually 0.014 inch in diameter.

In one embodiment, a piece of signal processing component can be interposed and embedded in the envelope of the proximal portion of the pressure wire such that a partially processed signal emerges on the continuation of a conductive trace.

In another embodiment, multiple such interposed segments can be implemented in the proximal portion of the pressure wire in order to reduce the size and weight of the transceiver 145 to better perform like a torque device.

In another embodiment, transceiver 145 only sends out the processed results for display without the pressure signals derived from the sensor chip 116.

The proximal portion of the pressure wire 110 is more tolerant of having any stiff sections that are required to implement signal conditioning and processing components. These components are being off-loaded from the torque device to enable a smaller form factor for the torque device that also doubles as a transceiver.

Note that this proximal portion of the pressure wire does not enter the human body.

In a modern catheterization laboratory, many pieces of equipment vie for the limited space available around the sterile patient table. Able to provide a minimally invasive pressure measurement device that conforms as much as possible to other interventional device like a balloon improves the work flow immensely.

As all the communication between the sensor chip and the torque device takes place in between the insulating layers and the conductive traces, the pressure sensing can also be implemented in the form of a stand-alone sleeve that is delivered over the preferred guide wire that the user has chosen.

This approach of performing the pressure measurement differs from the approach of implementing a pressure wire. The advantage of this approach is that the operator can use his preferred guide wire without any possible compromise on the wire performance but with the possible disadvantage that an additional catheter, however small, needs to be delivered over the guide wire and subsequently removed to allow for other device to be delivered over the same guide wire again for the next steps in the procedure.

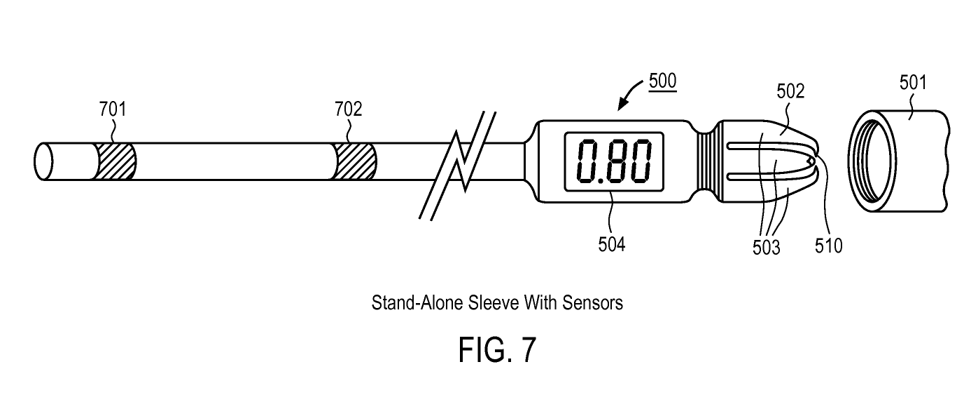

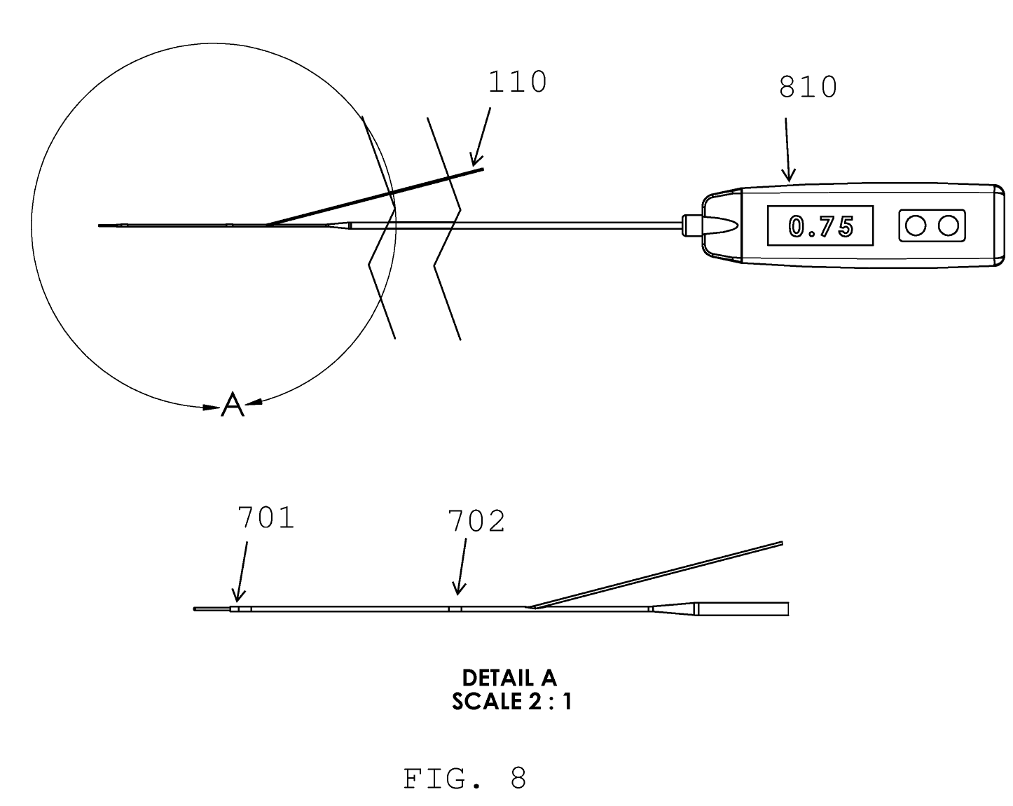

FIG. 7 illustrates the concept of this embodiment where sensor 701 and sensor 702 are located on a sleeve and are in communication, wireless or wired, with torque device 500. A display 504 is also shown on the torque device 500. This torque device 500 can also optionally communicate, via a wireless receive 146, with equipment 150 with its display 154 and input device 156 or a stand alone remote display.

In one embodiment, sensors 701 and 702 are wireless. Sensor 701 is distal to a stenosis in a coronary artery, sensor 702 is in the aorta. Together, they provide two independent pressure measurements that are transmitted to the torque device 500. The display, 504, on the torque device can then, as an example, display the measured Fractional Flow Reserve value which is a ratio of the mean of the distal pressure over the mean of the proximal pressure.

In one embodiment, the torque device 500 itself can activate the two sensors, 701 and 702, as indicated in FIG. 7. Sensor 701 is deployed distal to a stenosis in the coronary artery while sensor 702 remains in the aorta such that upon activation by the torque device via an electromagnetic wave, they send out their respective pressure measurement signals wirelessly. These signals are received by the torque device and any computation result based on these two measurement signals is then shown on the display 504. No other capital equipment in required and both pressure signals needed to generate the ratio for Fractional Flow Reserve (FFR) is obtained simultaneously without the need for a pullback.

It is also possible to implement sensor using MicroElectroMechanical Systems (MEMS) technology and they can be piezo-resistive or capacitive in their principle of operation. It is also possible to implement the sensor using piezo-electric polymer or ceramic.

The use of piezo-electric polymer is of particular value since it does not require the use of rigid sensor chip and can be conformable to the shape of a guide wire geometry.

The choice of the specific sensor technology for 701 and 702 depending on process complexity and cost of manufacturing with corresponding pro's and con's.

It should be appreciated that it is possible to have a hybrid system where the sensors 701 and 702 can have wired connections between them and then wirelessly communicate with torque device via wireless means. This has a certain advantage when the pressure sensing is implemented as a stand alone device to be delivered over an existing guide wire. Sensor 702 which resides in the aorta as opposed to the coronary artery would have more room to accommodate a wireless transceiver to transmit both pressure measurements. This will then not impact the need to have a small form factor in the distal sensor 701 to have accurate pressure measurement.

In one embodiment, the sensor 701 is implemented with a piezo-electric polymer that generates a voltage when experience a change in pressure. The capacitance of sensor 701 can also be a function of pressure as it changes dimension. This voltage or capacitance change is measured via conductive traces or other wired transmission means to a proximal sensor 702 which resides in the aorta. Sensor 702 itself senses pressure at the aorta as well as handling any needed conditioning and processing of pressure signal from sensor 701 and together wirelessly provides the result or partial result to the torque device 500 on its display 504.

It is contemplated that this invention is applicable to physiological parameters other than pressure. One characteristics of this invention is the use of a low cost, disposable transceiver. It can be made small if the data rate and power consumption are low—which dictates the kind of information and type of signal acquisition and processing that can be accomplished.

Physiologic parameters like pressure, temperature, pH value, etc., are slow varying parameters that can be acquired with low sampling frequency, simple processing, if any, and low data transmission rate. The power consumption is also correspondingly low.

The improvement described here affords a better torque transmission as it removes the need to have a lumen to accommodate the electrical or optical transmission lines. In particular, the electrical connection scheme also improves the electrical performance as the parasitic capacitance is reduced by increasing the separation of the transmission lines. The improved construction also allows for better integration of multiple sensors.

The improvement with a wireless transfer of the physiologic signal allows for a more compatible operation with how a guide wire is used in the PCI procedure. A wireless embodiment also improves the work flow and avoids the need to have a large instrument near the patient's bed during the procedure. Wireless communication between the sensor and the torque device also makes for a compact system when a simple display on the torque device is adequate for the procedure.

Multiple sensors eliminate the need to perform a pullback procedure to obtain pressure information from multiple locations.

A stand alone embodiment allows pressure measurement with an existing primary guide wire and eliminates the need for a wire exchange procedure.

Several variations of the stand alone sleeve with multiple sensors as illustrated by FIG. 7 are possible. For example, the distance between the two sensors, 701 and 702, can be made variable to accommodate different lesion locations in the coronary arteries while keeping the proximal sensor in the aorta.

The sleeve can also be constructed such that a guide wire exit port allows for a rapid exchange catheter configuration as described in U.S. Pat. No. 5,451,233 “Angioplasty Apparatus Facilitating Rapid Exchanges” by Paul Yock.

The sleeve in the above configuration can now have a catheter handle, as opposed to a torque device, where a larger display can be accommodated. This larger display can display both waveforms and numerical results from processing of the waveforms.

In this configuration, as shown in FIG. 8, the connection between the sensors (701, 702) and the electronics in the handle, 810, will not require embedding the conductors in insulating layers and are self contained within the stand-alone sleeve catheter.

Having the sensors implemented on the sleeve itself allows for integration with other interventional devices that could benefit from a pressure measurement to monitor the progress of the interventional procedure. For example, if this pressure measuring sleeve is integrated with a Chronic Total Occlusion (CTO) device, the pressure monitoring can indicate when the CTO device has succeeded in entering the distal true lumen as opposed to entering a false lumen in the intima of the vessel wall. This can reduces the use contrast medium and radiation from the angiogram.

Other applications can include integration with percutaneous valve implantation where the reduction of the pressure gradient across the valve is an important parameter. Having a sleeve approach for pressure measurement allows for relatively easy integration with such percutaneous valve devices.

In sum, the present invention provides a system and methods for an improved pressure measurement device. The advantages of such a system include the ability to manipulate the pressure wire more like a guide wire and perform the pressure measurement in a way more compatible with other catheterization laboratory procedures.

While this invention has been described in terms of several embodiments, there are alterations, modifications, permutations, and substitute equivalents, which fall within the scope of this invention. Although sub-section titles have been provided to aid in the description of the invention, these titles are merely illustrative and are not intended to limit the scope of the present invention.

It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A measuring system for measuring at least one physiological parameter of a human, the measurement system comprising:
a guide wire configured to be threaded into a vascular pathway of a human;
a sensor operatively coupled to a distal end of the guide wire, and wherein the sensor is configured to measure at least one physiological parameter inside the human; and
at least two transmission lines located on the guide wire surface for electrically coupling the sensor to a connector operatively coupled to the proximal end of the guide wire, and wherein the measured physiological parameter can be transmitted via the connector to a non-sterile equipment for display or further processing and display.

2. The measuring system of claim 1 wherein the connector includes a wireless transmitter or transceiver for transmitting the measured parameter to a corresponding transceiver or receiver coupled to a display or to another piece of equipment for further processing and display.

3. The measuring system of claim 1 wherein the connector includes a wireless transmitter and is adequately light weight such that it can be attached onto the guide wire in substantially arbitrary location along a proximal portion of the guide wire thereby enabling the connector to function like a torque device for manipulating the guide wire inside the vascular pathway of the human.

4. The measuring system of claim 1 wherein the connector is further configured to display the measured parameter.

5. The measuring system of claim 1 wherein the sensor is a Polyvinylidene Fluoride (PVDF) polymer sensor, or a Polyvinylidene Fluoride-co-Polytrifluoroethylene P(VDF-TrFE) copolymer sensor.

6. A measuring system for measuring at least one physiological parameter of a human, the measurement system comprising:
a guide wire configured to be threaded into a vascular pathway of a human;
at least two sensors operatively coupled to a distal end of the guide wire, and wherein the at least two sensors are configured to measure at least two physiological parameters at at least two corresponding locations inside the human; and
a connector operatively coupled to the proximal end of the guide wire, and wherein the connector is configured to receive the at least two measured parameters from the at least two sensors, and further configured to transmit the at least two measured parameters to a non-sterile equipment for display or further processing and display.

7. The measuring system of claim 6 further comprising at least three transmission lines located on the guide wire, and wherein the transmission lines are configured to electrically couple the at least two sensors to the connector.

8. The measuring system of claim 6 further comprising a low-power wireless transmitter located adjacent to at least one of the at least two sensors, and wherein the transmitter is configured to transmit the at least two measured parameters to the connector.

9. The measuring system of claim 6 wherein the connector is further configured to display the measured parameter.

10. The measuring system of claim 6 wherein one of the at least two sensors is a Polyvinylidene Fluoride (PVDF) polymer sensor, or a Polyvinylidene Fluoride-co-Polytrifluoroethylene P(VDF-TrFE) copolymer sensor.

11. A measuring system for measuring at least one physiological parameter of a human, the measurement system comprising:
an elongated sleeve configured to be delivered over a standard guide wire configured to be threaded into a vascular pathway of a human;
at least one sensor operatively coupled to a distal end of the sleeve, and wherein the at least one sensor is configured to measure at least one physiological parameter at at least one location inside the human; and
a connector operatively coupled to the proximal end of the sleeve, and wherein the connector is configured to receive the at least one measured parameter from the at least one sensor, and display the result of processing from the at least one parameter from the at least one location.

12. The measuring system of claim 11 wherein the sleeve has a guide wire exit port that is proximal to the at least one sensor and allows the sleeve to operate in a rapid-exchange configuration.

13. The measuring system of claim 11 wherein the connector which is operatively coupled to the proximal end of the sleeve functions also as a handle and a display

14. The measuring system of claim 11 wherein the sleeve is integrated with Chronic Total Occlusion crossing wire such that the display of the measured parameter indicates the status of the crossing of the occlusion.

15. The measuring system of claim 11 wherein the elongated sleeve includes a channel shaped and sized to travel along the guide wire as the sleeve is delivered into the vascular pathway.

16. The measuring system of claim 11 further comprising a low-power wireless transmitter located adjacent to the at least one sensor, and wherein the transmitter is configured to transmit the at least one measured parameter to the connector.

17. The measuring system of claim 11 wherein the connector is further configured to function as a torque device.

18. The measuring system of claim 11 wherein the connector is further configured to transmit the result of the processing or the at least one parameter to a remote display or a non-sterile equipment for display or further processing and display.

19. The measuring system of claim 11 wherein the at least one sensor is a piezo-electric sensor, a Polyvinylidene Fluoride (PVDF) polymer sensor, or a Polyvinylidene Fluoride-co-Polytrifluoroethylene P(VDF-TrFE) copolymer sensor.

20. A measuring system for measuring at least one physiological parameter of a human, the measurement system comprising:
an elongated sleeve configured to be delivered over a standard guide wire configured to be threaded into a vascular pathway of a human;
at least two sensors operatively coupled to a distal end of the sleeve, and wherein the at least two sensors are configured to measure at least two physiological parameters at at least two locations inside the human; and
a connector operatively coupled to the proximal end of the sleeve, and wherein the connector is configured to receive the at least two measured parameters from the at least two sensors.

21. The measuring system of claim 20 wherein the connector is further configured to display the result of processing from the at least two physiological parameters from the at least two locations.

22. The measuring system of claim 20 wherein the at least two sensors can have a separation that is adjustable.

23. The measuring system of claim 20 wherein the sleeve has a guide wire exit port that is proximal to the at least two sensors or between the at least two sensors and allows the sleeve to operate in a rapid-exchange configuration.

24. The measuring system of claim 20 wherein the connector which is operatively coupled to the proximal end of the sleeve catheter functions also as a handle and a display

25. The measuring system of claim 20 wherein one of the at least two sensors is coupled to a low power transmitter configured to transmit one of the at least two physiological parameters to the connector.

26. The measuring system of claim 20 wherein the connector is further configured to transmit one of the at least two physiological parameters to a remote display or a non-sterile equipment for display or further processing and display.

27. The measuring system of claim 20 wherein the connector is further configured to transmit the result of the processing or one of the at least two physiological parameters to a remote display or a non-sterile equipment for display or further processing and display.

28. The measuring system of claim 20 wherein one of the at least two sensors is a piezo-electric sensor, a Polyvinylidene Fluoride (PVDF) polymer sensor, or a Polyvinylidene Fluoride-co-Polytrifluoroethylene P(VDF-TrFE) copolymer sensor.
